(19) United States
(12) Patent Application Publication
(10) Pub. No.: US 2023/0158064 A1
(43) Pub. Date: May 25, 2023

(54) USE OF INHALED NITRIC OXIDE (iNO) FOR TREATMENT OF INFECTION, INCLUDING INFECTION WITH SARS-CoV2 AND TREATMENT OF COVID-19

(71) Applicant: BELLEROPHON THERAPEUTICS, Warren, NJ (US)

(72) Inventor: Parag SHAH, Morristown, NJ (US)

(73) Assignee: BELLEROPHON THERAPEUTICS, Warren, NJ (US)

(21) Appl. No.: 17/917,209

(22) PCT Filed: Apr. 6, 2021
(86) PCT No.: PCT/US2021/025950

§ 371 Date: Oct. 5, 2022

Related U.S. Application Data
(60) Provisional application No. 63/006,692, filed on Apr. 7, 2020.
(60) Provisional application No. 63/026,558, filed on May 18, 2020.

(51) Int. Cl. A61K 33/00; A61K 9/00; A61P 31/14; A61B 5/08

(52) U.S. Cl. CPC A61K 33/00; A61K 9/007; A61P 31/14; A61B 5/0816

(57) ABSTRACT

The present disclosure relates to use of pulsed dose inhaled nitric oxide for treatment of infection, including infection with SARS-CoV2 and the disease state COVID-19.

### CROSS-REFERENCE TO RELATED APPLICATIONS

The present application claims the benefit of U.S. Provisional Patent Application Nos. 63/006,692, filed Apr. 7, 2020, and 63/026,558, filed May 18, 2020, the entire contents of all of which are incorporated by reference herein in their entireties.

### FIELD OF THE INVENTION

The present application relates generally to methods for administration of nitric oxide, in particular, pulsatile delivery of nitric oxide to patients in need of therapeutic treatment of symptoms relating to infection, including infection with SARS-CoV2 and its associated disease state, COVID-19.

### BACKGROUND OF THE INVENTION

Nitric oxide (NO) is a gas that, when inhaled, acts to dilate blood vessels in the lungs, improving oxygenation of the blood and reducing pulmonary hypertension. Because of this, nitric oxide is provided as a therapeutic gas in the inspiratory breathing phase for patients having shortness of breath (dyspnea) due to a disease state, for example, pulmonary arterial hypertension (PAH), chronic obstructive pulmonary disease (COPD), combined pulmonary fibrosis and emphysema (CPFE), cystic fibrosis (CF), idiopathic pulmonary fibrosis (IPF), emphysema, interstitial lung disease (ILD), chronic thromboembolic pulmonary hypertension (CTEPH), chronic high altitude sickness, or other lung disease.

Inhaled nitric oxide (iNO) is a well-established safe and effective vasodilator and has been approved for the treatment of persistent pulmonary hypertension in neonates. As disclosed herein, pulse dosing utilizes high concentration pulses to ensure a precise and constant dose regardless of a patient's respiratory rate or inspiratory volume. The pulsatile technology allows us to titrate the dose, allowing much higher doses/concentrations than currently available in hospital based systems, as well as reduces the overall size of the therapy, allowing it to be administered at home.

While NO may be therapeutically effective when administered under the appropriate conditions, it can also become toxic if not administered correctly. NO reacts with oxygen to form nitrogen dioxide (NO₂), and NO₂ can be formed when oxygen or air is present in the NO delivery conduit. NO₂ is a toxic gas which may cause numerous side effects, and the Occupational Safety & Health Administration (OSHA) provides that the permissible exposure limit for general industry is only 5 ppm. Thus, it is desirable to limit exposure to NO₂ during NO therapy.

Coronaviruses are a family of viruses that can cause varying respiratory illnesses such as the common cold, SARS, and MERS, at various degrees of illness. The SARS-CoV2 virus (also originally known as n-CoV-19), was reported in December 2019 as originating in Wuhan, China, and is a strain of coronavirus that causes coronavirus disease 2019, or COVID-19. Symptoms of SARS-CoV2 infection/COVID-19 include, fever, cough, shortness of breath, and difficulty breathing. Some infected individuals lost the ability to smell and/or taste. Other symptoms may include body aches, pneumonia, chills, fatigue, nausea, diarrhea, and cold-like symptoms such as a runny nose or a sore throat. COVID-19 symptoms can range from mild to severe, and may lead to death, in part, due to complications caused by COVID-19, such as pneumonia and/or organ failure. On the other hand, some people infected with SARS-CoV2 may be asymptomatic. The incubation period for SARS-CoV2 ranges from one to fourteen days, with a median period from five to six days.

The clinical spectrum of the COVID-19 infection ranges from mild signs of upper respiratory tract infection to severe pneumonia and death. Currently, the probability of progression to end stage disease is not well understood; however, preventing progression in patients with mild or moderate disease can likely improve morbidity/mortality and reduce the impact on limited healthcare resources. Furthermore, reducing the need for positive pressure ventilator support as observed in Chen (2004) may limit lung damage. Based on the genomic similarities between the two coronaviruses, the data in SARS-CoV supports the potential for iNO to provide benefit for patients infected with COVID-19. Exogenous iNO in patients who have mild to moderate COVID-19 could prevent further deterioration and potentially improve the time to recovery.

No targeted therapeutic treatments for coronavirus (COVID-19) have been identified. Symptoms range from mild upper respiratory tract infection to severe pneumonia and death. Progression of end stage disease is unpredictable with high fatality rates in mechanically ventilated patients as a result of multi-organ failure. Prevention of COVID-19 progression in spontaneously breathing patients with mild to moderate disease may result in improved morbidity and mortality as well as limiting the burden to limited healthcare resources.

Nitric oxide plays a key role in suppressing viral replication. NO is a naturally produced molecule during the immune response to pathogens, with endogenous NO production upregulated by macrophages as a defense mechanism against some infections including bacterial, viral and protozoal. In vitro studies have shown that NO inhibits the replication of the severe acute respiratory syndrome-related coronavirus (SARS-CoV) (Akerstrom, et al, J. of Virology, 79, 2005, 1966-1969) and improves cellular survival of cells infected with SARS-CoV (Keyaerts et al, Int. J. of Infectious Disease, 8, 2004, 223-226). In a clinical study of SARS patients, iNO demonstrated improvements in arterial oxygenation, reduction in supplemental oxygen and need for ventilatory support. There were also improvements in chest radiography with a reduction in density of lung infiltrates (Chen, et al, Clinical Infectious Disease, 39, 2004, 1531-1535). Although the sample size was small, there appeared to be a shorter time to hospital discharge for those patients in the iNO group compared to controls.

The present invention is directed to using inhaled nitric oxide in a pulsed delivery system to treat symptoms of infection, in particular, viral infection with SARS-CoV2.

### SUMMARY OF THE INVENTION

In an embodiment of the present invention, a method for treating COVID-19 in a patient is taught, wherein, the method comprises administering a therapeutically effective amount of inhaled nitric oxide to the patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein the COVID-19 disease state is treated.

In an embodiment of the invention, a method for treating a viral, bacterial, or protozoal infection, which infection leads to development of a disease state in a patient is taught. The method comprises administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein the viral, bacterial, or protozoal infection is treated.

In an embodiment of the invention, the viral infection is SARS-CoV2 and the disease state is COVID-19.

In an embodiment of the invention, a method for inhibiting viral replication of SARS-CoV2 virus in a patient is taught. The method comprises administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein viral replication of SARS-CoV2 is inhibited.

In an embodiment of the invention, a method for reducing the need for supplemental oxygen in a patient suffering from a SARS-CoV2 infection or COVID-19 is taught. The method comprises administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein the need for supplemental oxygen is reduced or eliminated.

In an embodiment of the invention, a method for improving oxygenation of a patient suffering from SARS-CoV2 infection or COVID-19 is taught. The method comprises administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein oxygenation is improved.

In an embodiment of the invention, a method for improving oxygen saturation of a patient suffering from SARS-CoV2 infection or COVID-19 is taught. The method comprises administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein oxygen saturation is improved.

In an embodiment of the invention, a method for providing supportive care to a patient in respiratory distress due to COVID-19 is taught. The method comprising administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein the patient's respiratory distress is improved.

In an embodiment of the invention, a method for reducing the time a patient suffering from SARS-CoV2 infection or COVID-19 is in need of mechanical breathing assistance is taught. The method comprises administering a therapeutically effective amount of inhaled nitric oxide to said patient by a) detecting a breath pattern in said patient including a total inspiratory time; b) correlating the breath pattern with an algorithm to calculate the timing of administration of the dose of nitric oxide; and c) administering the dose of nitric oxide to said patient in a pulsatile manner over a portion of the total inspiratory time, wherein the time in need of mechanical breathing assistance is reduced or eliminated.

In an embodiment of the methods of the present invention, delivery of the dose of nitric oxide occurs within the first half of the total inspiratory time.

In an embodiment of methods of the present invention, the nitric oxide is delivered in a series of pulses over a period of time.

In an embodiment of the methods of the present invention, the inhaled nitric oxide is administered at a dose in a range of about 75 mcg/kg IBW/hr to about 200 mcg/kg IBW/hr. In an embodiment of the methods of the present invention, the inhaled nitric oxide is administered at a dose in a range of about 100 mcg/kg IBW/hr to about 150 mcg/kg IBW/hr. In an embodiment of the methods of the present invention, the inhaled nitric oxide is administered at a dose of about 125 mcg/kg IBW/hr.

In an embodiment of the methods of the present invention, the nitric oxide is administered in combination with at least one additional gas. In one embodiment, the at least one additional gas is oxygen.

In an embodiment of the methods of the present invention, the method further comprising the administration of at least one additional therapeutic agent.

In an embodiment of the methods of the present invention, administration of the iNO occurs in an outpatient setting.

In an embodiment of the methods of the present invention, the inhaled nitric oxide is administered for at least 24 hours per day over the course of the treatment period. In one embodiment, the inhaled nitric oxide is administered for least 18 hours per day over the course of the treatment period. In one embodiment, the inhaled nitric oxide is administered for least 12 hours per day over the course of the treatment period. In one embodiment, the inhaled nitric oxide is administered for least 8 hours per day over the course of the treatment period.

In an embodiment of the methods of the present invention, the treatment period is at least twenty-one days. In one embodiment, the treatment period is at least fourteen days. In one embodiment, the treatment period is at least ten days. In one embodiment, the treatment period is at least seven days. In one embodiment, the treatment period is at least five days. In one embodiment, the treatment period is five days or less. In one embodiment, the treatment period is four days or less. In one embodiment, the treatment period is three days or less. In one embodiment, the treatment period is two days or less. In one embodiment, the treatment period is one day or less.

In an embodiment of the present invention, a method for delivery of a dose of nitric oxide to a patient in need is taught. The method comprises a) detecting a breath pattern in said patient including a total inspiratory time using a device comprising a breath sensitivity control; b) correlating the breath pattern with an algorithm to calculate the timing of administration of the dose of nitric oxide, wherein the dose is from about 500 mcg/kg IBW/hr to about 1200 mcg/kg IBW/hr; and c) delivering the dose of nitric oxide to said patient in a pulsatile manner over a portion of the total inspiratory time.

In an embodiment of the invention, the dose is from about 500 mcg/kg IBW/hr to about 1000 mcg/kg/IBW. In an embodiment, the dose is 1000 mcg/kg IBW/hr. In one embodiment, the dose is 1050 mcg/kg IBW/hr.

In an embodiment of the invention, the dose of iNO is delivered once a day. In an embodiment of the invention, the dose of iNO is delivered twice a day. In an embodiment, the dose of iNO is delivered three times a day. In another embodiment, the dose of iNO is delivered four times a day. In another embodiment, the dose of iNO is delivered five times a day. In another embodiment, the dose of iNO is delivered two to four times a day.

In an embodiment of the invention, the dose of iNO is administered for at least 15 minutes per day over the course of the treatment period. In another embodiment, the dose of iNO is administered for at least 30 minutes per day over the course of the treatment period. In another embodiment, the dose of iNO is administered for at least 45 minutes per day over the course of the treatment period. In another embodiment, the dose of iNO is administered for at least one hour per day over the course of the treatment period. In another embodiment, the dose of iNO is administered for at least 1.5 minutes per day over the course of the treatment period. In another embodiment, the dose of iNO is administered for at least two hours per day over the course of the treatment period. In another embodiment, the dose of iNO is administered for between one to two hours per day over the course of the treatment period.

In an embodiment of the method of the present invention, the treatment period is from about one day to about seven days. In an embodiment, of the method of the present invention, the treatment period is one week to four weeks. In an embodiment, of the method of the present invention, the treatment period is two weeks. In an embodiment, of the method of the present invention, the treatment period is three weeks. In an embodiment, of the method of the present invention, the treatment period is four weeks.

Various embodiments are listed above and will be described in more detail below. It will be understood that the embodiments listed may be combined not only as listed below, but in other suitable combinations in accordance with the scope of the invention.

The foregoing has outlined rather broadly certain features and technical advantages of the present invention. It should be appreciated by those skilled in the art that the specific embodiments disclosed may be readily utilized as a basis for modifying or designing other structures or processes within the scope present invention. It should also be realized by those skilled in the art that such equivalent constructions do not depart from the spirit and scope of the invention as set forth in the appended claims.

### BRIEF DESCRIPTION OF THE DRAWINGS

The foregoing summary, as well as the following detailed description of the invention, will be better understood when read in conjunction with the appended drawings.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 represents an illustration of an embodiment of the treatment paradigm for COVID-19 (Siddiqi, et al., J. of Heart and Lung Transplantation, 2020, DOI: 10.1016/j.healun.2020.03.012).

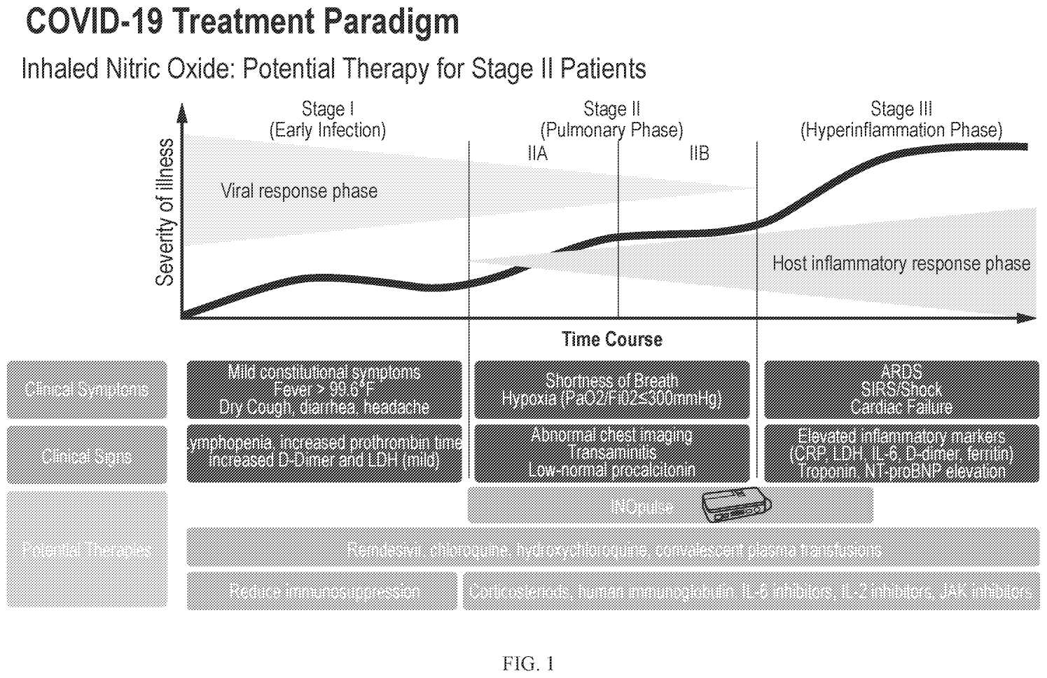

FIG. 2 represents an exemplary embodiment of a clinical study protocol according to the present invention.

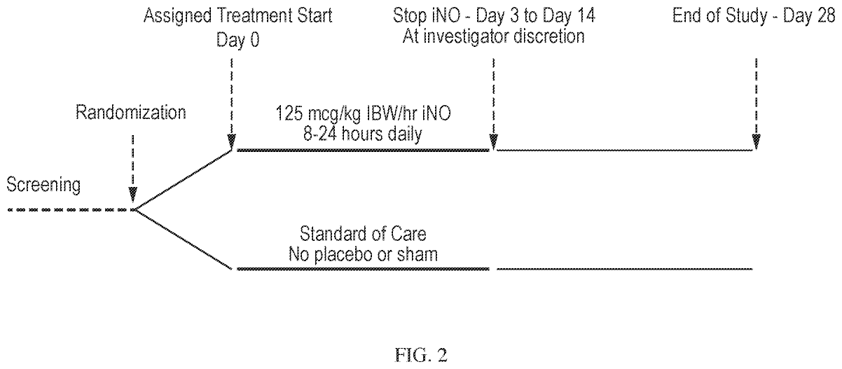

FIG. 3 represents an exemplary embodiment of a patient disposition schematic according to the present invention.

FIG. 4 is a representation of a patient timeline. Each line represents each patient's timeline starting from the time of hospitalization. Each bar within each line represents oxygen support after iNO treatment (mechanical ventilation (MV), non-invasive ventilation (NIV), high flow oxygen up to 60 L/min; low flow oxygen up to 16 L/min, or ambient air. All patients were on low flow oxygen during iNO treatment. Some patients were hospitalized before treatment with iNO. Black circles indicate deaths. Open diamonds indicate discharge from the hospital.

FIG. 5 represents a box and whiskers plot for oxygen therapy usage at the beginning of iNO treatment, the end of iNO treatment, post-iNO treatment, and at discharge for the patient study identified in Example 2. The box represents interquartile range and median; error bars represent maximum and minimum oxygen therapy.

FIG. 6 represents a box and whiskers plot for the SpO2 to FiO2 ratio at the beginning of iNO treatment, the end of iNO treatment, post-iNO treatment, and at discharge for the patient study identified in Example 2. The box represents interquartile range and median; error bars represent maximum and minimum oxygen therapy. FiO2 was estimated by assuming that the fraction of oxygen inspired (above normal atmospheric level or 20%) increased by 4% for every liter of oxygen flow administered.

FIG. 7 represents a box and whiskers plot for the 8-point ordinal scale at the beginning of iNO treatment, the end of iNO treatment, post-iNO treatment, and at discharge for the patient study identified in Example 2. The box represents interquartile range and median; error bars represent maximum and minimum oxygen therapy. The post-iNO measurement is taken the first day off of iNO.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

### Definitions

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe an aspect of the present invention, for example, dosing ranges, amounts of a component of a formulation, etc., all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Effective dosing of NO is based on a number of different variables, including quantity of drug and the timing of delivery. Several patents have been granted relating to NO delivery, including U.S. Pat. Nos. 7,523,752; 8,757,148; 8,770,199; and 8,803,717, and a Design Pat. D701,963 for a design of an NO delivery device, all of which are herein incorporated by reference. Additionally, there are pending applications relating to delivery of NO, including US2013/0239963 and US2016/0106949, both of which are herein incorporated by reference. Finally, there are pending application relating to the pulsed delivery of iNO, International PCT Application No. PCT/US2019/032887 and International PCT Application No. PCT/US2019/045806, which are herein incorporated by reference, that are relevant to methods for delivery of iNO according to the present invention. The present invention relates to use of these improved iNO delivery systems to address symptoms relating to infection, including infection with SARS-CoV2 and development of the COVID-19 disease state.

These improved delivery systems embodied, for example, in International PCT Application No. PCT/US2019/032887 and International PCT Application No. PCT/US2019/045806, include delivering a dose of a gas (e.g., NO) in a pulse to a patient during an inspiration by the patient. NO delivery can be precisely and accurately delivered within the first two-thirds of total breath inspiration time and the patient obtains benefits from such delivery. Such delivery minimizes loss of drug product and risk of detrimental side effects increases the efficacy of a pulse dose which in turn results in a lower overall amount of NO that needs to be administered to the patient in order to be effective. Such precision has further advantages in that only portions of the poorly ventilated lung area is exposed to NO. Hypoxia and issues with hemoglobin may also be reduced with such pulsed delivery, while NO₂ exposure is also more limited. Such delivery is useful for the treatment of various diseases, such as but not limited to idiopathic pulmonary fibrosis (IPF), pulmonary arterial hypertension (PAH), including Groups I-V pulmonary hypertension (PH), chronic obstructive pulmonary disorder (COPD), cystic fibrosis (CF), and emphysema, and is also useful as an antimicrobial, for example, in treating pneumonia or non-tuberculosis mycobacterium. It has been found that delivery of NO in this manner is also useful for treatment of symptoms of SARS-CoV2 infection, or COVID-19.

Delivery of iNO using this pulsatile method is also useful when administering high doses of iNO, e.g., 250 mcg/kg IBW/hr to 1200 mcg/kg IBW/hr, over a short period of time, e.g., 15 minutes to 4 hours.

### Breath Patterns, Detection and Triggers

As described in International PCT Application No. PCT/US2019/032887 and International PCT Application No. PCT/US2019/045806, for example, breath patterns vary based on the individual, time of day, level of activity, and other variables; thus it is difficult to predetermine a breath pattern of an individual. A delivery system that delivers therapeutics to a patient based on breath pattern, then, should be able to handle a range of potential breath patterns in order to be effective.

In certain embodiments, the patient or individual can be any age, however, in more certain embodiments the patient is sixteen years of age or older.

In an embodiment of the invention, the breath pattern includes a measurement of total inspiratory time, which as used herein is determined for a single breath. However, depending on context “total inspiratory time” can also refer to a summation of all inspiratory times for all detected breaths during a therapy. Total inspiratory time may be observed or calculated. In another embodiment, total inspiratory time is a validated time based on simulated breath patterns.

In an embodiment of the invention, breath detection includes at least one and in some embodiments at least two separate triggers functioning together, namely a breath level trigger and/or a breath slope trigger.

In an embodiment of the invention, a breath level trigger algorithm is used for breath detection. The breath level trigger detects a breath when a threshold level of pressure (e.g., a threshold negative pressure) is reached upon inspiration.

In an embodiment of the invention, a breath slope trigger detects breath when the slope of a pressure waveform indicates inspiration. The breath slope trigger is, in certain instances, more accurate than a threshold trigger, particularly when used for detecting short, shallow breaths.

In an embodiment of the invention, a combination of these two triggers provides overall a more accurate breath detection system, particularly when multiple therapeutic gases are being administered to a patient simultaneously.

In an embodiment of the invention, the breath sensitivity control for detection of either breath level and/or breath slope is fixed. In an embodiment of the invention, the breath sensitivity control for detection of either breath level or breath slope is adjustable or programmable. In an embodiment of the invention, the breath sensitivity control for either breath level and/or breath slope is adjustable from a range of least sensitive to most sensitive, whereby the most sensitive setting is more sensitive at detecting breaths than the least sensitive setting.

In certain embodiments where at least two triggers are used, the sensitivity of each trigger is set at different relative levels. In one embodiment where at least two triggers are used, one trigger is set a maximum sensitivity and another trigger is set at less than maximum sensitivity. In one embodiment where at least two triggers are used and where one trigger is a breath level trigger, the breath level trigger is set at maximum sensitivity.

Oftentimes, not every inhalation/inspiration of a patient is detected to then be classified as an inhalation/inspiration event for the administration of a pulse of gas (e.g., NO). Errors in detection can occur, particularly when multiple gases are being administered to a patient simultaneously, e.g., NO and oxygen combination therapies.

Embodiments of the present invention, and in particular an embodiment which incorporates a breath slope trigger alone or in combination with another trigger, can maximize the correct detection of inspiration events to thereby maximize the effectiveness and efficiency of a therapy while also minimizing waste due to misidentification or errors in timing.

In certain embodiments, greater than 50% of the total number of inspirations of a patient over a timeframe for gas delivery to the patient are detected. In certain embodiments, greater than 75% of the total number of inspirations of a patient are detected. In certain embodiments, greater than 90% of the total number of inspirations of a patient are detected. In certain embodiments, greater than 95% of the total number of inspirations of a patient are detected. In certain embodiments, greater than 98% of the total number of inspirations of a patient are detected. In certain embodiments, greater than 99% of the total number of inspirations of a patient are detected. In certain embodiments, 75% to 100% of the total number of inspirations of a patient are detected.

### Timing of a Pulse of NO

In an embodiment of the invention, the breath pattern is correlated with an algorithm to calculate the timing of administration of a dose of nitric oxide.

The precision of detection of an inhalation/inspiration event also permits the timing of a pulse of gas (e.g., NO) to maximize its efficacy by administering gas at a specified time frame of the total inspiration time of a single detected breath.

In an embodiment of the invention, at least fifty percent (50%) of the pulse dose of a gas is delivered over the first third of the total inspiratory time of each breath. In an embodiment of the invention, at least sixty percent (60%) of the pulse dose of a gas is delivered over the first third of the total inspiratory time. In an embodiment of the invention, at least seventy-five percent (75%) of the pulse dose of a gas is delivered over the first third of the total inspiratory time for each breath. In an embodiment of the invention, at least eighty-five (85%) percent of the pulse dose of a gas is delivered over the first third of the total inspiratory time for each breath. In an embodiment of the invention, at least ninety percent (90%) of the pulse dose of a gas is delivered over the first third of the total inspiratory time. In an embodiment of the invention, at least ninety-two percent (92%) of the pulse dose of a gas is delivered over the first third of the total inspiratory time. In an embodiment of the invention, at least ninety-five percent (95%) of the pulse dose of a gas is delivered over the first third of the total inspiratory time. In an embodiment of the invention, at least ninety-nine (99%) of the pulse dose of a gas is delivered over the first third of the total inspiratory time. In an embodiment of the invention, 90% to 100% of the pulse dose of a gas is delivered over the first third of the total inspiratory time.

In an embodiment of the invention, at least seventy percent (70%) of the pulse dose is delivered to the patient over the first half of the total inspiratory time. In yet another embodiment, at least seventy-five percent (75%) of the pulse dose is delivered to the patient over the first half of the total inspiratory time. In an embodiment of the invention, at least eighty percent (80%) of the pulse dose is delivered to the patient over the first half of the total inspiratory time. In an embodiment of the invention, at least 90 percent (90%) of the pulse dose is delivered to the patient over the first half of the total inspiratory time. In an embodiment of the invention, at least ninety-five percent (95%) of the pulse dose is delivered to the patient over the first half of the total inspiratory time. In an embodiment of the invention, 95% to 100% of the pulse dose of a gas is delivered over the first half of the total inspiratory time

In an embodiment of the invention, at least ninety percent (90%) of the pulse dose is delivered over the first two-thirds of the total inspiratory time. In an embodiment of the invention, at least ninety-five percent (95%) of the pulse dose is delivered over the first two-thirds of the total inspiratory time. In an embodiment of the invention, 95% to 100% of the pulse dose is delivered over the first two-thirds of the total inspiratory time.

When aggregated, administration of a number of pulse doses over a therapy session/timeframe can also meet the above ranges. For example, when aggregated greater than 95% of all the pulse doses administered during a therapy session were administered over the first two thirds of all of the inspiratory times of all of the detected breaths. In higher precision embodiments, when aggregated greater than 95% of all the pulse doses administered during a therapy session were administered over the first third of all of the inspiratory times of all of the detected breaths.

Given the high degree of precision of the detection methodologies of the present invention, a pulse dose can be administered during any specified time window of an inspiration. For example, a pulse dose can be administered targeting the first third, middle third or last third of a patient's inspiration. Alternatively, the first half or second half of an inspiration can be targeted for pulse dose administration. Further, the targets for administration may vary. In one embodiment, the first third of an inspiration time can be targeted for one or a series of inspirations, where the second third or second half may be targeted for one or a series of subsequent inspirations during the same or different therapy session. Alternatively, after the first quarter of an inspiration time has elapsed the pulse dose begins and continues for the middle half (next two quarters) and can be targeted such that the pulse dose ends at the beginning of the last quarter of inspiration time. In some embodiments, the pulse may be delayed by 50, 100, or 200 milliseconds (ms) or a range from about 50 to about 200 milliseconds.

The utilization of a pulsed dose during inhalation reduces the exposure of poorly ventilated areas of the lung and alveoli from exposure to a pulsed dose gas, e.g., NO. In one embodiment, less than 5% of poorly ventilated (a) areas of the lung or (b) alveoli are exposed to NO. In one embodiment, less than 10% of poorly ventilated (a) areas of the lung or (b) alveoli are exposed to NO. In one embodiment, less than 15% of poorly ventilated (a) areas of the lung or (b) alveoli are exposed to NO. In one embodiment, less than 20% of poorly ventilated (a) areas of the lung or (b) alveoli are exposed to NO. In one embodiment, less than 25% of poorly ventilated (a) areas of the lung or (b) alveoli are exposed to NO. In one embodiment, less than 30% of poorly ventilated (a) areas of the lung or (b) alveoli are exposed to NO. In one embodiment, less than 50% of poorly ventilated (a) areas of the lung or (b) alveoli are exposed to NO. In one embodiment, less than 60% of poorly ventilated (a) areas of the lung or (b) alveoli are exposed to NO. In one embodiment, less than 70% of poorly ventilated (a) areas of the lung or (b) alveoli are exposed to NO. In one embodiment, less than 80% of poorly ventilated (a) areas of the lung or (b) alveoli are exposed to NO. In one embodiment, less than 90% of poorly ventilated (a) areas of the lung or (b) alveoli are exposed to NO.

### Dosages and Dosing Regimens

In an embodiment of the invention, nitric oxide delivered to a patient is formulated at concentrations of about 3 to about 18 mg NO per liter, about 6 to about 10 mg per liter, about 3 mg NO per liter, about 6 mg NO per liter, or about 18 mg NO per liter. The NO may be administered alone or in combination with an alternative gas therapy. In certain embodiments, oxygen (e.g., concentrated oxygen) can be administered to a patient in combination with NO.

In an embodiment of the present invention, a volume of nitric oxide is administered (e.g., in a single pulse) in an amount of from about 0.350 mL to about 7.5 mL per breath. In some embodiments, the volume of nitric oxide in each pulse dose may be identical during the course of a single session. In some embodiments, the volume of nitric oxide in some pulse doses may be different during a single timeframe for gas delivery to a patient. In some embodiments, the volume of nitric oxide in each pulse dose may be adjusted during the course of a single timeframe for gas delivery to a patient as breath patterns are monitored. In an embodiment of the invention, the quantity of nitric oxide (in ng) delivered to a patient for purposes of treating or alleviating symptoms of a pulmonary disease on a per pulse basis (the “pulse dose”) is calculated as follows and rounded to the nearest nanogram value:

Dose mcg/kg-IBW/hr×Ideal body weight in kg (kg-IBW)×((1 hr/60 min)×(1 min/respiratory rate (bpm))×(1,000 ng/mcg).

As an example, Patient A at a dose of 100 mcg/kg IBW/hr has an ideal body weight of 75 kg, has a respiratory rate of 20 breaths per minute (or 1200 breaths per hour):

100 mcg/kg-IBW/hr×75 kg×(1 hr/1200 breaths)×(1,000 ng/ug)=6250 ng per pulse

In certain embodiments, the 60/respiratory rate (ms) variable may also be referred to as the Dose Event Time. In another embodiment of the invention, a Dose Event Time is 1 second, 2 seconds, 3 seconds, 4 seconds, 5 seconds, 6 seconds, 7 seconds, 8 seconds, 9 seconds, or 10 seconds.

In an embodiment of the invention, the iNO is administered at anywhere from 10 mcg/kg ideal body weight (IBW)/hr to 245 mcg/kg IBW/hr or more. In one embodiment, the iNO is administered from about 20 mcg/kg IBW/hr to about 150 mcg/kg IBW/hr. In one embodiment, the iNO is administered from about 25 mcg/kg IBW/hr to about 100 mcg/kg IBW/hr. In one embodiment, the iNO is administered from about 30 mcg/kg IBW/hr to about 75 mcg/kg IBW/hr. In one embodiment, the iNO is administered from about 25 mcg/kg IBW/hr to about 50 mcg/kg IBW/hr. In one embodiment, the iNO is administered from about 30 mcg/kg IBW/hr to about 45 mcg/kg IBW/hr. In one embodiment, the iNO is administered at 25 mcg/kg IBW/hr. In one embodiment, the iNO is administered at 30 mcg/kg IBW/hr. In one embodiment, the iNO is administered at 35 mcg/kg IBW/hr. In one embodiment, the iNO is administered at 40 mcg/kg IBW/hr. In one embodiment, the iNO is administered at 45 mcg/kg IBW/hr. In one embodiment, the iNO is administered at 50 mcg/kg IBW/hr. In one embodiment, the iNO is administered at 55 mcg/kg IBW/hr. In one embodiment, the iNO is administered at 60 mcg/kg IBW/hr. In one embodiment, the iNO is administered at 65 mcg/kg IBW/hr. In one embodiment, the iNO is administered at 70 mcg/kg IBW/hr. In one embodiment, the iNO is administered at 75 mcg/kg IBW/hr. In one embodiment, the iNO is administered at 80 mcg/kg IBW/hr. In one embodiment, the iNO is administered at 85 mcg/kg IBW/hr. In one embodiment, the iNO is administered at 90 mcg/kg IBW/hr. In one embodiment, the iNO is administered at 95 mcg/kg IBW/hr. In one embodiment, the iNO is administered at 100 mcg/kg IBW/hr. In one embodiment, the iNO is administered at 105 mcg/kg IBW/kg. In one embodiment, the iNO is administered at 110 mcg/kg IBW/hr. In one embodiment, the iNO is administered at 115 mcg/kg IBW/hr. In one embodiment, the iNO is administered at 120 mcg/kg IBW/hr. In one embodiment, the iNO is administered at 125 mcg/kg IBW/hr. In one embodiment, the iNO is administered at 130 mcg/kg IBW/hr. In one embodiment, the iNO is administered at 135 mcg/kg IBW/hr. In one embodiment, the iNO is administered at 140 mcg/kg IBW/hr. In one embodiment, the iNO is administered at 145 mcg/kg IBW/hr. In one embodiment, the iNO is administered at 150 mcg/kg IBW/hr. In one embodiment, the iNO is administered at 155 mcg/kg IBW/hr. In one embodiment, the iNO is administered at 160 mcg/kg IBW/hr. In one embodiment, the iNO is administered at 165 mcg/kg IBW/hr. In one embodiment, the iNO is administered at 170 mcg/kg IBW/hr. In one embodiment, the iNO is administered at 175 mcg/kg IBW/hr. In one embodiment, the iNO is administered at 180 mcg/kg IBW/hr. In one embodiment, the iNO is administered at 185 mcg/kg IBW/hr. In one embodiment, the iNO is administered at 190 mcg/kg IBW/hr. In one embodiment, the iNO is administered at 195 mcg/kg IBW/hr. In one embodiment, the iNO is administered at 200 mcg/kg IBW/hr. In one embodiment, the iNO is administered at 205 mcg/kg IBW/hr. In one embodiment, the iNO is administered at 210 mcg/kg IBW/hr. In one embodiment, the iNO is administered at 215 mcg/kg IBW/hr. In one embodiment, the iNO is administered at 220 mcg/kg IBW/hr. In one embodiment, the iNO is administered at 225 mcg/kg IBW/hr. In one embodiment, the iNO is administered at 230 mcg/kg IBW/hr. In one embodiment, the iNO is administered at 235 mcg/kg IBW/hr. In one embodiment, the iNO is administered at 240 mcg/kg IBW/hr. In one embodiment, the iNO is administered at 245 mcg/kg IBW/hr.

In an embodiment of the invention, the iNO is administered at anywhere from 250 mcg/kg ideal body weight (IBW)/hr to 1200 mcg/kg IBW/hr or more. In one embodiment, the iNO is administered from about 300 mcg/kg IBW/hr to about 1100 mcg/kg IBW/hr. In one embodiment, the iNO is administered from about 400 mcg/kg IBW/hr to about 1000 mcg/kg IBW/hr. In one embodiment, the iNO is administered from about 500 mcg/kg IBW/hr to about 1050 mcg/kg IBW/hr. In one embodiment, the iNO is administered at 250 mcg/kg IBW/hr. In one embodiment, the iNO is administered at 275 mcg/kg IBW/hr. In one embodiment, the iNO is administered at 300 mcg/kg IBW/hr. In one embodiment, the iNO is administered at 325 mcg/kg IBW/hr. In one embodiment, the iNO is administered at 350 mcg/kg IBW/hr. In one embodiment, the iNO is administered at 375 mcg/kg IBW/hr. In one embodiment, the iNO is administered at 400 mcg/kg IBW/hr. In one embodiment, the iNO is administered at 425 mcg/kg IBW/hr. In one embodiment, the iNO is administered at 450 mcg/kg IBW/hr. In one embodiment, the iNO is administered at 475 mcg/kg IBW/hr. In one embodiment, the iNO is administered at 500 mcg/kg IBW/hr. In one embodiment, the iNO is administered at 525 mcg/kg IBW/hr. In one embodiment, the iNO is administered at 550 mcg/kg IBW/hr. In one embodiment, the iNO is administered at 575 mcg/kg IBW/hr. In one embodiment, the iNO is administered at 600 mcg/kg IBW/hr. In one embodiment, the iNO is administered at 625 mcg/kg IBW/hr. In one embodiment, the iNO is administered at 650 mcg/kg IBW/hr. In one embodiment, the iNO is administered at 675 mcg/kg IBW/hr. In one embodiment, the iNO is administered at 700 mcg/kg IBW/hr. In one embodiment, the iNO is administered at 725 mcg/kg IBW/hr. In one embodiment, the iNO is administered at 750 mcg/kg IBW/hr. In one embodiment, the iNO is administered at 725 mcg/kg IBW/hr. In one embodiment, the iNO is administered at 750 mcg/kg IBW/hr. In one embodiment, the iNO is administered at 775 mcg/kg IBW/hr. In one embodiment, the iNO is administered at 800 mcg/kg IBW/hr. In one embodiment, the iNO is administered at 825 mcg/kg IBW/hr. In one embodiment, the iNO is administered at 850 mcg/kg IBW/hr. In one embodiment, the iNO is administered at 875 mcg/kg IBW/hr. In one embodiment, the iNO is administered at 900 mcg/kg IBW/hr. In one embodiment, the iNO is administered at 925 mcg/kg IBW/hr. In one embodiment, the iNO is administered at 950 mcg/kg IBW/hr. In one embodiment, the iNO is administered at 975 mcg/kg IBW/hr. In one embodiment, the iNO is administered at 1000 mcg/kg IBW/hr. In one embodiment, the iNO is administered at 1025 mcg/kg IBW/hr. In one embodiment, the iNO is administered at 1050 mcg/kg IBW/hr. In one embodiment, the iNO is administered at 1075 mcg/kg IBW/hr. In one embodiment, the iNO is administered at 1100 mcg/kg IBW/hr. In one embodiment, the iNO is administered at 1125 mcg/kg IBW/hr. In one embodiment, the iNO is administered at 1150 mcg/kg IBW/hr. In one embodiment, the iNO is administered at 1175 mcg/kg IBW/hr. In one embodiment, the iNO is administered at 1200 mcg/kg IBW/hr.

In an embodiment of the invention, the patient is also administered oxygen with the iNO. In an embodiment of the invention, the oxygen is administered at up to 20 L/minute. In an embodiment of the invention, the oxygen is administered at up to 1 L/minute, 2 L/minute, 3 L/minute, 4 L/minute, 5 L/minute, 6 L/minute, 7 L minute, 8 L/minute, 9 L/minute, 10 L/minute, 11 L/minute, 12 L/minute, 13 L/minute, 14 L/minute, 15 L/minute, 16 L/minute, 17 L/minute, 18 L/minute, 19 L/minute, or 20 L/minute. In an embodiment of the invention, oxygen is administered as prescribed by a physician.

In an embodiment of the invention, a single pulse dose provides a therapeutic effect (e.g., a therapeutically effective amount of NO) to the patient. In another embodiment of the invention, an aggregate of two or more pulse doses provides a therapeutic effect (e.g., a therapeutically effective amount of NO) to the patient.

In an embodiment of the invention, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 675, about 700, about 750, about 800, about 850, about 900, about 950, or about 1000 pulses of nitric oxide is administered to a patient every hour.

In an embodiment of the invention, a nitric oxide therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment, a nitric oxide therapy session occurs over a timeframe of about 10 minutes to about 5 hours. In an embodiment, the timeframe is about 10 minutes, 15 minutes, 20 minutes, 25 minutes, 30 minutes, 35 minutes, 40 minutes, 45 minutes, 50 minutes 55 minutes, 60 minutes, 65 minutes, 70 minutes, 75 minutes, 80 minutes, 85 minutes, 90 minutes, 105 minutes, 120 minutes, 135 minutes, 150 minutes, 165 minutes, 180 minutes, 195 minutes, 210 minutes, 225 minutes, 240 minutes, 255 minutes, 270 minutes, 285 minutes, or 300 minutes. In an embodiment, the timeframe is about 15 minutes to about 3 hours, about 30 minutes to about 2.5 hours, about 1 hour to about 2 hours, or about 2 hours to 3 hours. In an embodiment, a nitric oxide therapy session occurs over a timeframe of about 15 minutes, 30 minutes, 45 minutes, 1 hour, 1.5 hours, 2 hours, 2.5 hours, 3 hours, 3.5 hours, 4 hours, 4.5 hours, or 5 hours.

In an embodiment of the invention, a nitric oxide treatment is administered for a timeframe of a minimum course of treatment. In an embodiment of the invention, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the invention, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the invention, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In an embodiment of the invention, a nitric oxide treatment session is administered one or more times per day. In an embodiment of the invention, nitric oxide treatment session may be once, twice, three times, four times, five times, six times, or more than six times per day. In an embodiment of the invention, the treatment session may be administered once a month, once every two weeks, once a week, once every other day, daily, or multiple times in one day.

### Administration of Oxygen

In an embodiment of the invention, oxygen is administered to the patient in accordance with instructions from a treating physician. In an embodiment of the invention, the oxygen is administered at up to 20 L/minute. In an embodiment of the invention, the oxygen is administered at up to 1 L/minute, 2 L/minute, 3 L/minute, 4 L/minute, 5 L/minute, 6 L/minute, 7 L minute, 8 L/minute, 9 L/minute, 10 L/minute, 11 L/minute, 12 L/minute, 13 L/minute, 14 L/minute, 15 L/minute, 16 L/minute, 17 L/minute, 18 L/minute, 19 L/minute, or 20 L/minute. In an embodiment of the invention, oxygen is administered as prescribed by a physician. In another embodiment, the patient is administered oxygen 24 hours per day. In another embodiment, the patient is administered oxygen for at least 1, 2, 3, 4, 5, 6, 7, 8, 9, 10, 11, 12, 13, 14, 15, 16, 17, 18, 19, 20, 21, 22, 23, or 24 hours per day. In another embodiment, the patient is administered oxygen for at least 12 hours per day.

### Methods of Treatment

In an embodiment of the invention, methods for treating an infection, including the SARS-CoV2 infection, a bacterial infection, or other viral infection are taught. In another embodiment, methods for treating symptoms of an infection or disease, including the SARS-CoV2 infection, COVID-19, a bacterial or viral infection are taught. In another embodiment, methods for improving oxygen saturation in a patient are taught. In another embodiment, methods for improving oxygenation in a patient are taught. In another embodiment, methods for reducing the requirement for oxygen therapy or reducing the amount of time a patient is on oxygen therapy are taught. In another embodiment, methods for reducing the need for or reducing the amount of time a patient is on mechanical breathing assistance, e.g., a ventilator or intubation, are taught. In another embodiment, a method of treating COVID-19 is taught. In another embodiment, a method for reducing the severity of respiratory symptoms associated with COVID-19 is taught. In another embodiment, a method for treating acute respiratory distress syndrome (ARDS) associated with COVID-19 is taught. In another embodiment, methods of use in an outpatient setting are taught.

The methods include administration of iNO in accordance with the dosing and dosing regimens discussed herein, and optionally supplementing iNO administration with oxygen. In an embodiment of the invention, iNO is administered according to the pulsed manner discussed herein. In an embodiment of the invention, the iNO is delivered to a patient using the INOpulse® device (Bellerophon Therapeutics).

In an embodiment of the invention, oxygenation in a patient is improved. In one embodiment, oxygenation is improved as compared with a baseline oxygenation level. In one embodiment, oxygenation is improved by about 1% to about 50%. In another embodiment, oxygenation is improved by about 1% to about 25%. In another embodiment, oxygenation is improved by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, oxygenation is improved by about 10%, 15%, 20%, 25%, 30%, 35%, 40%, 45%, or 50%.

In another embodiment of the invention, oxygenation is maintained as compared with a baseline oxygenation level. In another embodiment, oxygenation is does not decrease as compared with a baseline oxygenation level. In another embodiment, oxygenation declines less over time in treated patients than untreated or placebo patients.

In an embodiment of the invention, oxygen saturation levels are improved. In one embodiment, the oxygen saturation levels are improved as compared with a baseline oxygen saturation level. In one embodiment, oxygen saturation levels are improved by about 1% to about 50%. In another embodiment, oxygen saturation levels are improved by about 1% to about 25%. In another embodiment, oxygen saturation levels are improved by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, oxygen saturation levels are improved by about 10%, 15%, 20%, 25%, 30%, 35%, 40%, 45%, or 50%.

In another embodiment of the invention, oxygen saturation levels are maintained as compared with a baseline oxygen saturation level. In another embodiment, oxygen saturation levels do not decrease as compared with a baseline oxygen saturation level. In another embodiment, oxygen saturation levels decline less over time in treated patients than untreated or placebo patients.

In an embodiment of the invention, the time a patient is on mechanical breathing assistance is reduced as compared to an untreated patient. In one embodiment, the time on mechanical breathing assistance is reduced by about 1% to about 50%. In another embodiment, the time on mechanical breathing assistance is reduced by about 1% to about 25%. In another embodiment, the time on mechanical breathing assistance is reduced by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, the time on mechanical breathing assistance is reduced by about 10%, 15%, 20%, 25%, 30%, 35%, 40%, 45%, or 50%. In another embodiment, treatment with iNO according to the present invention avoids the need for mechanical breathing assistance.

In an embodiment of the invention, the time a patient is on supplemental oxygen therapy is reduced as compared to an untreated patient. In one embodiment, the time on supplemental oxygen therapy is reduced by about 1% to about 50%. In another embodiment, the time on supplemental oxygen therapy is reduced by about 1% to about 25%. In another embodiment, the time on supplemental oxygen therapy is reduced by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, the time on supplemental oxygen therapy is reduced by about 10%, 15%, 20%, 25%, 30%, 35%, 40%, 45%, or 50%. In another embodiment, treatment with iNO according to the present invention avoids the need for supplemental oxygen therapy.

In an embodiment of the invention, reduction of the severity of respiratory symptoms associated with a viral, bacterial, or protozoal infection and disease states associated therewith, including, for example, SARS-CoV2 and COVID-19, occurs with treatment of iNO according to the present invention. In one embodiment, reduction of severity of respiratory symptoms occurs with a 75 mcg/kg IBW/hr to about 200 mcg/kg IBW/hr dose of iNO delivered in a pulsatile manner for a period of up to 24 hours daily, over a period of about seven to fourteen days. In one embodiment, reduction of severity of respiratory symptoms occurs with a 100 mcg/kg IBW/hr to about 150 mcg/kg IBW/hr dose of iNO delivered in a pulsatile manner for a period of up to 24 hours daily, over a period of about seven to ten days. In one embodiment, reduction of severity of respiratory symptoms occurs with a 125 mcg/kg IBW/hr dose of iNO delivered in a pulsatile manner for a period of up to 24 hours daily, over a period of about seven to fourteen days.

In an embodiment of the invention, the dosing regimen is about 125 mcg/kg IBW/hr of iNO for a period of up to 24 hours daily, for a period of about one day, two days, three days, four days, five days, six days, or seven days, and up to fourteen days, depending on the clinical necessity for the iNO.

In an embodiment of the invention, the iNO is administered in an outpatient setting to avoid the need for a patient to be admitted to the hospital, or if already hospitalized, to lessen the time required to be in a hospital setting. Such an outpatient setting can be the patient's home, a clinic, or an ambulatory environment.

### Administration of Other Therapeutic Agents

In an embodiment of the invention, iNO is administered before, concurrently with, or after, another therapeutic agent. In an embodiment, a therapeutically effective amount of another therapeutic agent is administered to a patient in need thereof to treat a bacterial or viral infection, or a disease caused by such a bacterial or viral infection. In one embodiment, the therapeutic agent is an anti-IL-6 antibody, hydroxychloroquine, chloroquine, favilar, remdesivir, a vaccine, an anti-inflammatory, a steroid (e.g., glucocorticoid such as prednisone, prednisolone, or methylprednisone) or a derivative or precursor thereof. In another embodiment, the therapeutic agent is an agent useful in treating respiratory disease, breathing difficulties, and/or pneumonia.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: An Adaptive, Randomized, Open Label Study to Assess the Efficacy and Safety of Pulsed, Inhaled Nitric Oxide (iNO) Versus Standard of Care (SOC) in Subjects with Mild or Moderate Coronavirus Disease (COVID-19)

This example discloses a randomized, open-label study to assess the efficacy and safety of pulsed iNO compared to standard of care (SOC) in subjects with COVID-19 who are hospitalized and require supplemental oxygen without assisted ventilation. Up to 500 subjects are randomized to receive either (a) iNO 125 mcg/kg IBW/hr for at least 8 hours and up to 24 hours daily for 3 days to 14 days or until resolution or discharge at the discretion of the Investigator, or (b) standard of care. No placebo arm is used. Subjects are followed through Day 28 to assess their clinical status. The primary objective in this study is to verify the efficacy of iNO in subjects with COVID-19. The secondary objective in this study is to evaluate the safety of iNO in subjects with COVID-19.

Outcomes are assessed using an outcome scale assessed 14 days after randomization. An example outcome scale is shown in Table A.

TABLE A
8-point Ordinal Outcome Scale, 14 Days post-randomization

| Score | Outcome |
|---|---|
| 1 | Death |
| 2 | Hospitalized, requiring mechanical ventilation or ECMO |
| 3 | Hospitalized, requiring non-invasive ventilation or high flow |
| | oxygen |
| 4 | Hospitalized, requiring supplemental oxygen |
| 5 | Hospitalized, not requiring supplemental oxygen - requiring |
| | ongoing medical care (COVID-19 related or otherwise) |
| 6 | Hospitalized, not requiring supplemental oxygen - not requiring |
| | ongoing medical care (COVID-19 related or otherwise). |
| 7 | Not hospitalized - limitation on activities and/or requiring home |
| | oxygen |
| 8 | Not hospitalized, no limitations on activities. |

Ordinal scale was collected daily for each subject from the start of iNO treatment until day of discharge or death.

Methodology

Prior to receiving treatment with iNO, subjects were screened to confirm eligibility. COVID-19 infection must have been confirmed via positive RT-PCR, or having a high suspicion of infection, with symptom onset within the previous 8 days. Upon successful completion of screening, subjects are randomized to receive treatment with iNO 125 mcg/kg IBW/hr versus standard of care for 3 to 14 days or until resolution or discharge at the discretion of the Investigator. Subjects are followed through about Day 28 to assess clinical status. Subjects in the iNO group are treated by means of an INOpulse device using a readily available cannula on site.

At day 0 of the study, subjects testing positive for COVID-19 are evaluated for vital signs, blood chemistry, and a chest x-ray or CT scan, and an overall physical exam is performed. Daily vital signs, oxygen measurements (pulse oximetry), methemoglobin levels, and adverse events are assessed through the entire course of treatment.

Upon discontinuation of iNO, subjects are monitored for symptoms of pulmonary rebound, which include hypoxemia, bradycardia, tachycardia, systemic hypotension, shortness of breath, near-syncope, and syncope. On the day of discharge, the subjects are provided another chest x-ray or CT scan, blood chemistry panel, COVID-19 test, and adverse event assessment.

About 1 month after initial dosing, subjects are contacted to assess vitals and adverse event(s).

Initial data for three COVID-19 patients that completed the treatment regimen demonstrated improved oxygenation, which allowed them to avoid the need for mechanical ventilation.

### Example 2: Pulsed Inhaled Nitric Oxide in Patients with Hypoxemia Due to COVID-19

Use of INOpulse was requested through the US Food and Drug Administration (FDA) individual expanded access program (IEAP). For each potential patient, treating physicians applied for and received authorization from the manufacturer (Bellerophon Therapeutics, Warren, N.J.) and then the FDA, and were assigned a unique Emergency Investigational New Drug (EIND) number as individual EIND sponsors; local IRB approval was also obtained for each patient. All patients completed an informed consent process for the use of an unapproved therapy. The program was overseen, and therapy was supplied by the manufacturer. The IEAP program was first granted authorization on Mar. 19, 2020.

Hospitalized patients with positive RT-PCR (reverse transcription polymerase chain reaction) for SARS-CoV-2 from a nasopharyngeal swab or with high index of suspicion of COVID-19 and needing supplemental oxygen via nasal cannula at no more than 10 L/min were eligible for the IEAP. Patients with left ventricular ejection fraction of less than 40% or a known history of left heart failure were ineligible. Oxygen flow requirements and oxygen saturations were collated from various time points throughout the hospital course. Measures were compared prior to and after starting iNO therapy. These measures were followed through the course of treatment and until the patient was discharged or had progressed to assisted ventilation. INOpulse therapy was provided in addition to the standard of care treatment for COVID-19 and was administered to patients under the supervision of the individual site investigators. INOpulse was set to deliver NO at 125 mcg/kg IBW/hour (equivalent to approximately 20 ppm) and was to be used for 8-24 hours per day, for up to 14 days, or until there was clinical improvement no longer requiring iNO. There were no pre-specified endpoints; however, data was collected on oxygen requirements, need for escalation of therapy (e.g. intubation) and duration of hospital stay. Using this data, we also collated an 8-point ordinal scale daily for each patient (Table A, above). This scale is currently being used in the Adaptive COVID-19 Treatment Trial (ACTT: https://clinicaltrials.gov/ct2/show/NCT04280705).

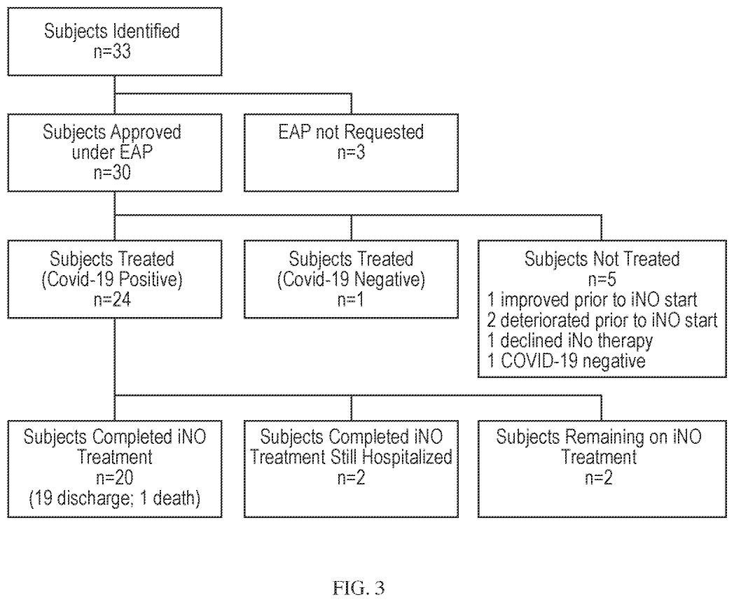

There were 25 patients treated with INOpulse therapy. Twenty-four were verified to be COVID-19 positive (FIG. 3). The mean age of the population was 57.8 (±14.4 SD) years, the majority were male and African American (Table B). Most patients had comorbidities and had been started on other investigational treatments for COVID-19.

TABLE B
Patient Demographics*

| Total Patients | N = 24 | |
|---|---|---|
| Age | 57.8 (±14.4 SD) | Years |
| Male Sex | 16 | (67%) |

Race

| | | |
|---|---|---|
| African American | 16 | (67%) |
| White | 7 | (29%) |
| Asian | 1 | (4%) |

Comorbidities

| | | |
|---|---|---|
| Hypertension | 12 | (50%) |
| Type 2 Diabetes Mellitus | 13 | (54%) |
| Hyperlipidemia | 4 | (17%) |
| Cardiovascular Disease | 5 | (21%) |
| Cancer | 3 | (13%) |

COVID-19 Investigational Treatments

| | | |
|---|---|---|
| Hydroxychloroquine + Azithromycin | 8 | (33%) |
| Hydroxychloroquine + Azithromycin + | 3 | (13%) |
| Tocilizumab | | |
| Sarilumab/Placebo + Azithromycin | 3 | (13%) |
| Convalescent Plasma | 2 | (8%) |
| Vit C + Zinc | 6 | (25%) |

Oxygen Flow & Saturations Pre-iNO

| | | |
|---|---|---|
| Oxygen Flow (median) | 5.5 | L/min |

| | |
|---|---|
| Saturations (median) | 93% |

iNO Therapy

| | | |
|---|---|---|
| iNO Treatment Duration (median, range) | 4 (2-9) | Days |
| Time to Discharge from start of iNO | 6 (3-14) | Days |
| (median, range) | | |
| Time to Discharge from hospitalization | 10 (6-23) | Days |
| (median, range) | | |

*Only patients that completed iNO treatment and discharged included in analysis of time to discharge.

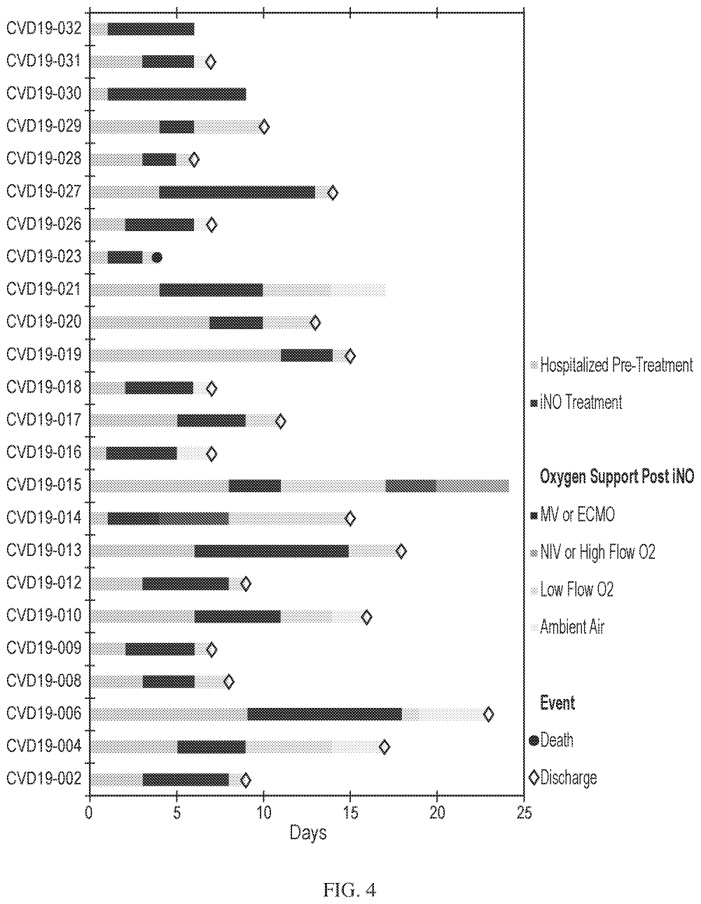

FIG. 4 demonstrates the time course in days of the events for each subject including hospitalization, oxygen therapy, INOpulse therapy, escalation of respiratory support, and discharge. There was one death in an elderly man with severe comorbidities who only received iNO for less than 24 hours towards the end of his disease course. In the subjects treated with INOpulse, the median oxygen flow requirements and oxygen saturations measured via pulse oximetry at the start of iNO therapy were 5 L/min and 94% respectively (Table C).

TABLE C
Patient Summary

| | Pre-iNO | iNO-Start | iNO-End | Post-iNO | Discharge |
|---|---|---|---|---|---|
| Patients (n) | 24 | 24 | 22 | 22 | 20 |

Oxygen Therapy (L/min)

| Median | 5.5 | 5.0 | 3.3 | 2.0 | 0.0 |
|---|---|---|---|---|---|
| IQR* | 6.3 | 4.5 | 2.0 | 0.5 | 1.0 |
| Min, Max | 2.0, 60.0 | 1.0, 16.0 | 1.0, 10.0 | 0.0, 10.0 | 0.0, 3.0 |

Oxygen Saturation (SpO2)

| | | | | | |
|---|---|---|---|---|---|
| Median | 93% | 94% | 95% | 95% | 95% |
| IQR | 3% | 2% | 5% | 4% | 2% |
| Min, Max | 88%, 98% | 91%, 97% | 89%, 100% | 89%, 100% | 90%, 100% |

8 Point Ordinal Scale

| Median | N/A | 4 | 4 | 4 | 7 |
|---|---|---|---|---|---|
| IQR | N/A | 0 | 0 | 3 | 1 |
| Min, Max | N/A | 3, 4 | 3, 4 | 1, 8 | 1, 8 |

*IQR = interquartile range; 8-point ordinal scale collected starting at time of iNO treatment; post-iNO ordinal scale represents first day off; one patient intubated post-iNO and one patient death at discharge censored from oxygen therapy and oxygen saturation analysis.

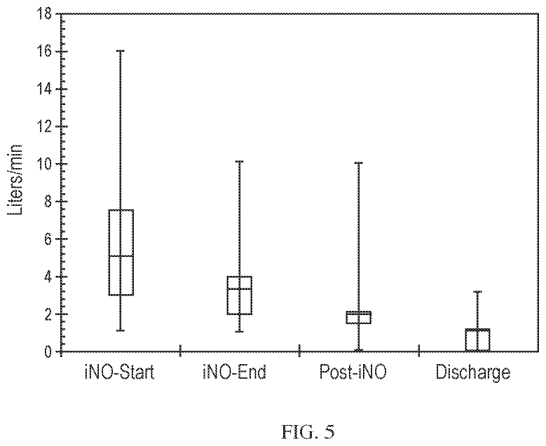

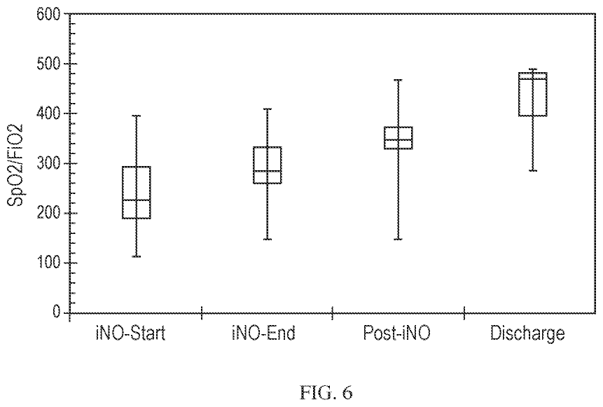

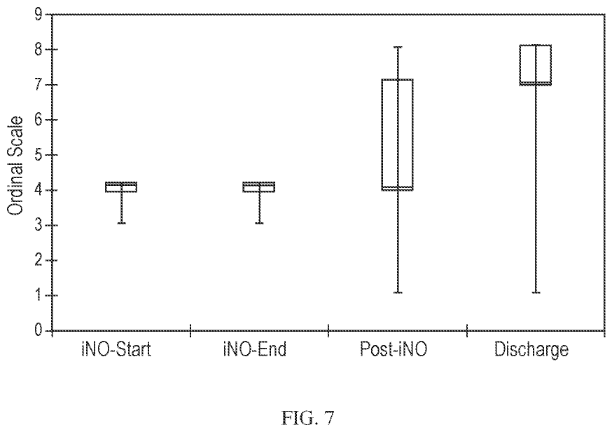

Most patients were treated with the iNO continuously for 24 hours. A box and whiskers plot of the oxygen flow requirements over time from the start of iNO treatment through discharge is provided (FIG. 5). Compared with baseline, the ratio of oxygen saturation to fraction of inspired oxygen (SpO2/FiO2) improved after the initiation of iNO from a median of 229 to 286 on completion, and to 346 in the period after iNO, consistent with improving oxygenation (FIG. 6). The median 8-point ordinal scores are shown for the group in Table 3. The median score improved from 4 at the initiation of INOpulse therapy to 7 (not hospitalized but with limitation of activities) at the time of discharge (FIG. 7). At the time of this report, 19 patients had been discharged from the hospital with the median time to discharge from the start of iNO treatment being 6 days (range 3-14 days).

INOpulse was generally well tolerated with no reports of increase in methemoglobin levels above 1.5%. There were 5 Serious Adverse Events (SAE) reported in 4 patients. The single case of death was in a 76-year-old man (CVD19-023) with poorly controlled type 2 diabetes mellitus (T2DM), hypertension, with a history of a recent fall with associated extensive skin infection, septicemic and in acute renal failure. He was started on iNO, improved initially but then deteriorated and iNO was withdrawn after approximately 14 hours of treatment when palliative care was initiated. The second patient was a 60-year-old male (CVD19-014) with T2DM and obesity who was started on iNO for 3 days but deteriorated requiring intubation and ventilation for 5 days. He subsequently improved, was extubated on Day 9 and weaned down to 1 L/min of oxygen with discharge home a few days later. The third patient (CVD19-015) was a 60-year-old male with chronic lymphocytic leukemia and acute on chronic renal failure who started on iNO for two days with improvement and the iNO was stopped. He worsened over the next few days with hypoxemia and seizures and was placed on mechanical ventilation on Day 10. He was extubated after 3 days and managed on high flow oxygen. The fourth patient was a 65-year-old African American male with a history of hypertension and stroke who started iNO with initial improvement in oxygenation, but later had worsening hypoxemia due to fluid overload and the iNO was stopped due to him requiring high flow oxygen. After achieving a negative fluid balance with diuretics, the iNO was re-initiated and he was ultimately weaned entirely from iNO and oxygen and discharged home.

One patient with suspected COVID-19 infection was treated with iNO for 3 days but tested negative on two occasions by RT-PCR and the iNO was therefore discontinued. His treatment course was uneventful, and he was eventually discharged from the hospital on day 18. He was not included in the analysis. One subject (CVD19-029) received a higher dose of iNO at 250 mcg/kg IBW/hour (equivalent to approximately 40 ppm). This higher dose was well tolerated, and the subject responded well and was discharged home without supplemental oxygen.

Most of the current investigational treatments for COVID-19 target either the virus or the immune response. Pulsed iNO targets the pulmonary vasculature as well as the virus. In this series of 24 patients with moderate to severe COVID-19 infection requiring oxygen therapy, treatment with INOpulse was followed by improvements in oxygenation with 19 having been discharged at the time of reporting. There was one death in a patient with multiple comorbidities and severe disease who had limited iNO exposure.

These data highlight the inherent poor outcomes in patients with COVID-19 placed on mechanical ventilation, albeit some of this high mortality might be attributable to an overwhelmed system. The complications of mechanical ventilation are well-known in terms of nosocomial infections, volutrauma, and other ventilator-related iatrogenesis. Indeed, an underappreciated aspect of having enough ventilators is the people power and skills necessary to manage them. This further serves to underscore the importance of avoiding mechanical ventilation where possible by strategies that might “buy time” and reduce the need for this resource intensive salvage strategy. One of the priorities of research therefore should be preventative measures to avert disease progression, thereby minimizing the risk and need for mechanical ventilation. On the other end of the spectrum, it is noteworthy that apart from the one patient who succumbed, nineteen of the remaining 23 patients were discharged home despite being on high amounts of supplemental oxygen at the initiation of iNO therapy. This raises the speculative concept of whether iNO hastens resolution of COVID pneumonia with reductions in hospital lengths of stay.

The ability to provide pulsed iNO outside of the ICU or hospital setting, would allow physicians to treat these patients early, targeting both the viral load as well as the pulmonary vasculature. If the benefits of iNO are confirmed in a randomized, controlled trial this would have important implications. A reduction in the number of patients requiring assisted ventilation would lower the demand on stressed ICU resources and if hospital length of stay can further be curtailed, this would enable the throughput of COVID-19 patients thereby freeing up capacity for new COVID-19 patients.

While preferred embodiments of the invention are shown and described herein, such embodiments are provided by way of example only and are not intended to otherwise limit the scope of the invention. Various alternatives to the described embodiments of the invention may be employed in practicing the invention.

We claim:

1. A method for treating COVID-19 in a patient, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein the COVID-19 is treated.

2. A method for treating a viral, bacterial, or protozoal infection leading to development of a disease state in a patient, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein the viral, bacterial, or protozoal infection is treated.

3. The method of claim 2, wherein the viral infection is SARS-CoV2 and the disease state is COVID-19.

4. A method for inhibiting viral replication of SARS-CoV2 virus in a patient, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein viral replication of SARS-CoV2 is inhibited.

5. A method for reducing the need for supplemental oxygen in a patient suffering from a SARS-CoV2 infection or COVID-19, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein the need for supplemental oxygen is reduced or eliminated.

6. A method for improving oxygenation of a patient suffering from SARS-CoV2 infection or COVID-19, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein oxygenation is improved.

7. A method for improving oxygen saturation of a patient suffering from SARS-CoV2 infection or COVID-19, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein oxygen saturation is improved.

8. A method for providing supportive care to a patient in respiratory distress due to COVID-19, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein the patient's respiratory distress is improved.

9. A method for reducing the time a patient suffering from SARS-CoV2 infection or COVID-19 is in need of mechanical breathing assistance, the method comprising administering a therapeutically effective amount of inhaled nitric oxide to said patient by:
a) Detecting a breath pattern in said patient including a total inspiratory time;
b) Correlating the breath pattern with an algorithm to calculate the timing of administration of the dose of nitric oxide; and
c) Administering the dose of nitric oxide to said patient in a pulsatile manner over a portion of the total inspiratory time,
wherein the time in need of mechanical breathing assistance is reduced or eliminated.

10. The method of any of claims 1-9, wherein delivery of the dose of nitric oxide occurs within the first half of the total inspiratory time.

11. The method of any of claims 1-9, wherein the nitric oxide is delivered in a series of pulses over a period of time.

12. The method of any of claims 1-9, wherein the inhaled nitric oxide is administered at a dose in a range of about 75 mcg/kg IBW/hr to about 200 mcg/kg IBW/hr.

13. The method of any of claims 1-9, wherein the inhaled nitric oxide is administered at a dose in a range of about 100 mcg/kg IBW/hr to about 150 mcg/kg IBW/hr.

14. The method of claim 13, wherein the inhaled nitric oxide is administered at a dose of about 125 mcg/kg IBW/hr.

15. The method of any of claims 1-14, wherein the nitric oxide is administered in combination with at least one additional gas.

16. The method of claim 15, wherein the at least one additional gas is oxygen.

17. The method of any of claim 15 or 16, further comprising the administration of at least one additional therapeutic agent.

18. The method of claim 17, wherein administration of the iNO occurs in an outpatient setting.

19. The method of claims 1-14, wherein the inhaled nitric oxide is administered for at least 24 hours per day over the course of the treatment period.

20. The method of claim 19, wherein the inhaled nitric oxide is administered for least 18 hours per day over the course of the treatment period.

21. The method of claim 20, wherein the inhaled nitric oxide is administered for least 12 hours per day over the course of the treatment period.

22. The method of claim 21, wherein the inhaled nitric oxide is administered for least 8 hours per day over the course of the treatment period.

23. The method of any of claims 19-22, wherein the treatment period is at least twenty-one days.

24. The method of claim 23, wherein the treatment period is at least fourteen days.

25. The method of claim 24, wherein the treatment period is at least ten days.

26. The method of claim 25, wherein the treatment period is at least seven days.

27. The method of claim 26, wherein the treatment period is at least five days.

28. A method for delivery of a dose of nitric oxide to a patient in need, said method comprising:
a) Detecting a breath pattern in said patient including a total inspiratory time using a device comprising a breath sensitivity control;
b) Correlating the breath pattern with an algorithm to calculate the timing of administration of the dose of nitric oxide, wherein the dose is from about 500 mcg/kg IBW/hr to about 1200 mcg/kg IBW/hr; and
c) Delivering the dose of nitric oxide to said patient in a pulsatile manner over a portion of the total inspiratory time.

29. The method of claim 28, wherein the dose is from about 500 mcg/kg IBW/hr to about 1000 mcg/kg/IBW.

30. The method of claim 28, wherein the dose is 1000 mcg/kg IBW/hr.

31. The method of claim 28, wherein the dose is 1050 mcg/kg IBW/hr.

32. The method of any of claims 28-31, wherein the dose of iNO is delivered twice a day.

33. The method of any of claims 28-31, wherein the dose of iNO is delivered three times a day.

34. The method of any of claims 28-31, wherein the dose of iNO is delivered four times a day.

35. The method of any of claims 28-31, wherein the dose of iNO is delivered five times a day.

36. The method of any of claims 28-31, wherein the dose of iNO is delivered two to four times a day.

37. The method of any of claims 32-36, wherein the dose of iNO is administered for at least 15 minutes per day over the course of the treatment period.

38. The method of any of claims 32-36, wherein the dose of iNO is administered for at least 30 minutes per day over the course of the treatment period.

39. The method of any of claims 32-36, wherein the dose of iNO is administered for at least 45 minutes per day over the course of the treatment period.

40. The method of any of claims 32-36, wherein the dose of iNO is administered for at least one hour per day over the course of the treatment period.

41. The method of any of claims 32-36, wherein the dose of iNO is administered for at least 1.5 minutes per day over the course of the treatment period.

42. The method of any of claims 32-36, wherein the dose of iNO is administered for at least two hours per day over the course of the treatment period.

43. The method of any of claims 32-36, wherein the dose of iNO is administered for between one to two hours per day over the course of the treatment period.

44. The method of any of claims 37-43, wherein the treatment period is from about one day to about seven days.
